(19) United States
(12) Patent Application Publication
(10) Pub. No.: US 2016/0317068 A1
(43) Pub. Date: Nov. 3, 2016

(54) Electronic devices with encapsulating silicone based adhesive

(71) Applicant: Verily Life Sciences LLC, Mountain View, CA (US)

(72) Inventors: Brian Marc Pepin, Oakland, CA (US); James Etzkorn, Mountain View, CA (US)

(21) Appl. No.: 15/095,670

(22) Filed: Apr. 11, 2016

Related U.S. Application Data
(60) Provisional application No. 62/155,319, filed on Apr. 30, 2015.

(51) Int. Cl. A61B 5/1477; H01L 21/56; A61B 5/00; H01L 23/29; H01L 23/00; H01L 21/48; H01L 23/31

(52) U.S. Cl. CPC A61B 5/1477; H01L 21/4853; H01L 21/56; H01L 23/3142; H01L 23/29; H01L 24/32; H01L 24/83; A61B 5/6821; H01L 2224/32501; H01L 2224/32221; H01L 2224/8338

(57) ABSTRACT

A flexible electronic device is provided that includes electronics, metal traces, and other components at least partially encapsulated in a protective, corrosion- and fluid-resistant encapsulating adhesive coating. The device include electronics, sensors, and other components disposed on a flexible substrate that is configured to be mounted to a body or disposed in some other environment of interest. The encapsulating adhesive coating is flexible and adheres securely to the electronics, metal traces, and other components disposed on the flexible substrate. The encapsulating adhesive coating prevents voids from forming proximate the components within which water or other chemicals could be deposited from the environment of the device. The encapsulating adhesive coating could include silicone or other flexible highly adhesive substances. The encapsulating adhesive coating could be a conformal coating.

### CROSS-REFERENCE TO RELATED APPLICATION

This application claims priority to U.S. Provisional Patent Application No. 62/155,319, filed Apr. 30, 2015, which is incorporated herein by reference.

### SUMMARY

Some embodiments of the present disclosure provide a method including: (i) forming traces in a layer of metal, wherein the layer of metal is disposed on an adhesive layer that is disposed on a flexible substrate, and wherein forming the traces includes patterning the layer of metal to provide electrical connections between one or more electronic components; (ii) disposing one or more electronic components on the traces, wherein disposing the one or more electronic components on the traces includes electrically connecting the one or more electronic components to the traces; and (iii) forming an encapsulating sealant layer that is configured to adhere to the adhesive layer and to at least partially encapsulate the traces and the one or more electronic components.

Some embodiments of the present disclosure provide a body-mountable device including: (i) a flexible substrate; (ii) an adhesive layer disposed on the flexible substrate; (iii) one or more electronic components disposed on the adhesive layer; (iv) metal traces disposed on the adhesive layer that provide electrical connections to the one or more electronic components; and (v) an encapsulating sealant layer that is configured to adhere to the adhesive layer and to at least partially encapsulate the metal traces and the one or more electronic components..

These as well as other aspects, advantages, and alternatives, will become apparent to those of ordinary skill in the art by reading the following detailed description, with reference where appropriate to the accompanying drawings.

### BRIEF DESCRIPTION OF THE DRAWINGS

FIG. 1A is a top aspect view of an example body-mountable device. FIG. 1B is a cross-sectional view of the example body-mountable device shown in FIG. 1A.

FIG. 2A is a cross-sectional view of an example body-mountable device during fabrication of the body-mountable device.

FIG. 2B is a cross-sectional view of the body-mountable device of FIG. 2A at a later stage in the fabrication of the body-mountable device.

FIG. 2C is a cross-sectional view of the body-mountable device of FIG. 2B at a later stage in the fabrication of the body-mountable device.

FIG. 2D is a cross-sectional view of the body-mountable device of FIG. 2C at a later stage in the fabrication of the body-mountable device.

FIG. 2E is a cross-sectional view of the body-mountable device of FIG. 2D at a later stage in the fabrication of the body-mountable device.

FIG. 3 is view of a number of body-mountable devices being fabricated in a roll-to-roll process.

FIG. 4 is a flowchart of an example method.

### DETAILED DESCRIPTION

In the following detailed description, reference is made to the accompanying figures, which form a part hereof. In the figures, similar symbols typically identify similar components, unless context dictates otherwise. The illustrative embodiments described in the detailed description, figures, and claims are not meant to be limiting. Other embodiments may be utilized, and other changes may be made, without departing from the scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

I. Overview

Some embodiments of the present disclosure provide a body-mountable device configured to be mounted to a skin surface or other location of a living body (e.g., to skin of the upper arm or abdomen of a person) and including electronics or other components (e.g., sensors) configured to provide functions of the body-mountable device. For example, the body-mountable device could be configured to detect a physiological parameter or property (e.g., a blood flow rate, a pulse rate, a blood oxygen saturation, a concentration of an analyte in blood or other fluids) of a body to which the body-mountable device is mounted at one or more points in time. The body-mountable device could additionally or alternatively include user interfaces (e.g., user inputs, displays, indicators), communications interfaces (e.g., RFID, Bluetooth), or other components to provide applications of the body-mountable device.

The body-mountable device can be configured to be flexible such that the body-mountable device minimally interferes with activities of a body to which the body-mountable device is mounted and/or such that the body-mountable device can be mounted to a body comfortably for protracted periods of time. For example, the body-mountable device could include a flexible substrate to which other components or elements of the body-mountable device are mounted (e.g., electronics, sensors, sealant layers) such that the flexible substrate and/or the body-mountable device as a whole complies with the shape of the skin surface and deforms with changes in the shape of the skin surface. Those of skill in the art will recognize that body-mountable devices as described herein may be provided in devices that could be mounted on a variety of portions of the human body to measure a variety of physiological properties of the human body (e.g., concentrations of a variety of analytes in a variety of fluids of the body, temperature, galvanic properties, ECG, muscle activity). Those of skill in the art will also recognize that the sensing platform described herein may be provided in devices that could be mounted in locations other than locations on a human body, e.g., locations on an animal body, locations that are part of a natural or artificial environment. Such a flexible substrate could be configured to be mounted to the skin surface (e.g., by use of glue, tape, dry adhesive, or other adhesive means). Alternatively, such a flexible substrate could be configured to be mounted to an eye, e.g., by being at least partially embedded in an ophthalmic lens or other structure (e.g., a hydrogel contact lens).

In some examples, such body-mountable devices could be operated when exposed to fluids (e.g., blood, sweat, tears, saliva, interstitial fluid, water or other fluids from the environment of a body). Such fluids could be conductive, corrosive, or could have other properties that could interfere with the operation of electronics exposed to such fluids (e.g., by providing a low-impedance path between metal traces of an electronic device). In such examples, a variety of coverings, seals, adhesives, or other materials or treatments could be included/applied to a body-mountable device to, e.g., prevent and/or control exposure of elements of the body-mountable device to the fluids. Such materials could be flexible. Such materials could be impermeable to the fluids and/or to constituents of the fluids (e.g., impermeable to water vapor). Additionally or alternatively, such materials could be applied to electronic components, metal interconnects, and/or other components of the body-mountable device to fully or partially encapsulate such elements. Such encapsulation could be provided to, e.g., prevent fluid from being transported through such materials to condense or otherwise form proximate to the electronic components, metal interconnects, and/or other components.

Such a flexible layer of sealant could be disposed in a variety of ways on electronic components (e.g., integrated circuits, sensors, light emitters, antennas), metallic traces, or other components of a body-mountable device to fully or partially encapsulate such components of the body-mountable device. For example, the flexible layer of sealant could be formed by chemical vapor deposition, spin-coating, spray-coating, inkjet printing, screen printing, or some other methods. Further, properties of precursor materials (e.g., monomer solutions) or other materials used to form such a sealant layer and/or methods used to form such a sealant layer could be specified to control properties of the formed sealant layer (e.g., to control a geometry mean thickness, depth, or other properties of the sealant layer). For example, such an encapsulating sealant layer could be a conformal sealant layer, e.g., could be disposed conformally across surfaces of electronics, metal traces, or other elements of a body-mountable device with a thickness that is substantially the same (e.g., that varies by less than 20%) across the encapsulated surfaces of such elements of the body-mountable device. In another example, such an encapsulating sealant layer could be a planarizing sealant layer, e.g., could be disposed to a uniform height (e.g., a height that varies by less than 20%) above an underlying adhesive layer or other substrate material on which the sealant layer, electronics, metal traces, or other components of the body-mountable device are disposed. Certain electronic elements or other components could protrude from such a planarizing sealant layer due to having heights that are greater than the uniform height of the planarizing sealant layer. Other geometries, thicknesses, heights, or other properties of an encapsulating sealant layer of a body-mountable device, as described herein, are possible.

In some examples, a formed layer of a sealant or other material could include one or more windows or other formed features to allow one or more sensors or other components of a body-mountable device access to a fluid to which the body-mountable device is exposed (e.g., interstitial fluid, sweat, tears, blood). In some examples, a body-mountable device could include multiple seals, adhesives, or other materials or treatments (e.g., a layer of flexible adhesive underlying traces and electronic components of the body-mountable device and a conformal, planarizing, or otherwise encapsulating layer disposed over the traces and electronics and that is adhered to the underlying layer).

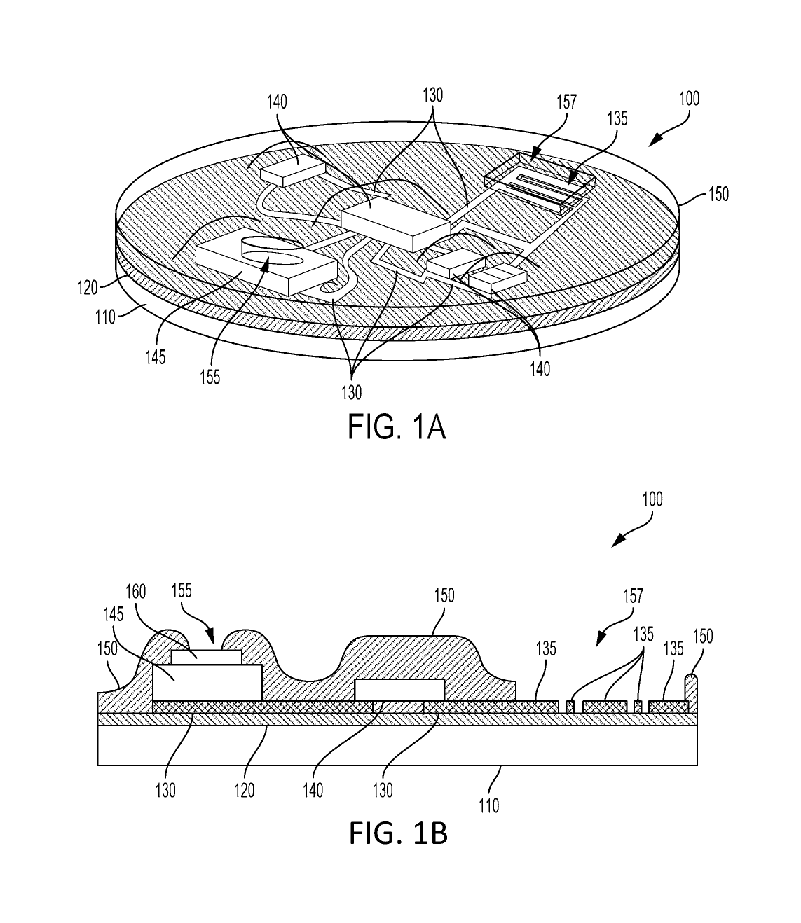

FIG. 1A illustrates an example body-mountable device 100. The body-mountable device 100 includes a flexible substrate 110. An adhesive layer 120 is disposed on the flexible substrate 110 and metal traces 130 are disposed on the adhesive layer 10 the metal traces 130 provide electrical connections between electronic components (e.g., 140, 145) of the body-mountable device 100. The metal traces 130 additionally include electrodes 135 that could be operated to electrochemically detect a property (e.g., a pH level, a concentration of an analyte) of a fluid to which the electrodes 135 are exposed. an encapsulating sealant layer 150 is formed to adhere to the electronics 140, 145, metal traces 130, and adhesive layer 120 such that the electronics 140, 145 and metal traces 130 are at least partially encapsulated within the encapsulating sealant layer 150 and the adhesive layer 120. The encapsulating sealant layer 150 includes windows 155, 157 that allow a fluid to which the body-mountable device 100 is exposed (e.g., tears, interstitial fluid, sweat, blood) to be accessed by the electronics 145 and the electrodes 135, respectively.

As illustrated in FIG. 1A, the body-mountable device 100 is formed as a circular patch. A body-mountable device could take other shapes, e.g., elongated shapes, rectangles, rings, or other shapes according to an application. In some examples, a body-mountable device could be formed to have a ring shape, an arc shape, or some other shape to facilitate mounting to an eye and/or embedding in an ophthalmic device, e.g., an ophthalmic lens. In some examples, a body-mountable device could be formed to have one or more protrusions or extensions according to an application. For example, a body-mountable device could be formed to include one or more elongate portions (e.g., a sensor probe) that could be configured to penetrate skin such that one or more sensors (e.g., electrochemical sensors) disposed on the elongate portions could contact interstitial fluid, blood, or other fluids within the skin. The body-mountable device could be formed to include one or more loops, straps, tabs, openings, slots, or other features configured to facilitate mounting of the device to a target portion of a body (e.g., by an adhesive, by a tape, by insertion within/around/between parts of a body). The body-mountable device could be configured to be mounted within a mouth or other cavity of a body, to be implanted within an enclosed body cavity (e.g., a peritoneal cavity, a cranial cavity), to be implanted within a tissue (e.g., to be emplaced within an incision formed in a tissue), or to be mounted on or within a body of a human or animal in some other way.

The flexible substrate 110 could be composed of polyimide or some other flexible polymeric or other material. The flexible substrate could have a thickness between approximately 5 microns and approximately 100 microns. Further, the flexible substrate 110 could have a size specified to minimally interfere with activities of a living body to which the device 100 is mounted. For example, the flexible substrate 110 could have size (e.g., a diameter of a circular portion, as illustrated in FIGS. 1A and 1B) less than approximately 11 millimeters. Diameter and thickness values are provided for explanatory purposes only. Further, the shape of the flexible substrate 110 could be different from that illustrated in FIGS. 1A and 1B or elsewhere herein; for example, the flexible substrate 110 could have an elongate shape, a square or rectangular shape, or some other shape according to an application. For example, the flexible substrate 110 could have an elongate shape to provide sufficient area for disposition of electronics, batteries, user interface components (e.g., touch sensor electrodes, flexible display elements), antennas, or other components on the flexible substrate 110 while minimally impeding motion and/or deformation of a skin or other tissue surface to which the flexible substrate 110 is mounted (e.g., by being formed and/or mounted to a skin surface such the orientation of the elongate shape of the flexible substrate 110 is perpendicular to a direction of strain of the skin surface).

FIG. 1B shows the body-mountable device 100 in cross-section. FIG. 1B additionally shows an analyte-sensitive material 160 disposed on a sensor 145 of the electronics. The analyte-sensitive material 160 is configured to selectively interact with (e.g., to selectively reversibly or irreversibly bind to, to selectively interact with, to selectively catalyze a reaction of) an analyte of interest (e.g., a sugar, a protein, a hormone, a cell, an ion, an antibody) within a fluid to which the body-mountable device 100 is exposed such that the sensor 145 can detect the analyte in the fluid. FIG. 1B shows that the encapsulating sealant layer 150 has been disposed to encapsulate, in combination with the adhesive layer 120, the metal traces 130 and the electronics 140, 145 except for areas exposed by the windows 155, 157. As a result, the body-mountable device 100 includes substantially no voids or volumes proximate to surfaces of the metal traces 130 and electronics 140, 145 (other than those surfaces exposed by the windows 155, 157) in which water vapor or other material (e.g., material that has permeated through the encapsulating sealant layer 150 from a fluid to which the body-mountable device 100 is exposed) could be deposited, condense, or otherwise accumulate.

As shown in FIG. 1B, the encapsulating sealant layer 150 is disposed substantially conformally over those surfaces of the body-mountable device 100 that are encapsulated by the encapsulating sealant layer 150. That is, the thickness of the encapsulating sealant layer 150 is substantially uniform across the surfaces on which the encapsulating sealant layer 150 is disposed. However, this is a non-limiting example of an encapsulating sealant layer of a body-mountable device as described herein. In another example, such an encapsulating sealant layer could be disposed to have a substantially uniform height relative to an underlying substrate and/or underlying adhesive layer (e.g., relative to the adhesive layer 120). Such an encapsulating sealant layer could be described as a planarizing sealant layer. Electronic components or other elements of a body-mountable device that includes such a planarizing sealant layer could be partially encapsulated by such a planarizing layer such that aspects of such components that have a height, relative to the underlying adhesive layer or other substrate, that is greater than the height of the planarizing layer are not encapsulated by the planarizing sealant layer (e.g., such portions could be exposed). For example, the height of such a planarizing sealant layer could be specified such that the analyte-sensitive material 160 is not encapsulated by the planarizing sealant layer (e.g., such that the analyte-sensitive material 160 is exposed to a fluid of interest in the environment of the body-mountable device 100).

The flexible substrate 110 could be formed from a variety of materials and/or combinations of materials. For example, the flexible substrate could include polyimide, polyethylene terephthalate (PET), a thermoplastic material (e.g., a liquid crystal polymer), or some other polymer or other flexible material according to an application. Further, the metal traces 130 could be formed from a variety of conductors and/or combinations of conductors, e.g., platinum, copper, aluminum, steel, silver, gold, tantalum, titanium, or some other materials. Additionally or alternatively, the metal traces 130 could include surface coatings to, e.g., increase a corrosion resistance of the metal traces 130 (e.g., gold, tantalum, titanium), to control a mechanical property of the metal traces 130, or according to some other consideration. In some examples, the metal traces 130 could have a specified thickness, e.g., between approximately 5 microns and approximately 25 microns. In some examples, one or more of the metal traces 130 and/or surface coatings thereof could be specified and/or treated to provide a specified electrochemical property, e.g., a specified electrode potential. For example, the electrodes 135 could be formed by applying a surface coating to the metal traces 130 or by performing some other method. For example, one of the electrodes 135 could include be a silver/silver-chloride electrode formed by depositing silver on the metal traces 130 and subsequently forming a layer of silver-chloride on the deposited silver (e.g., by applying a current through the electrode while exposing the electrode to a chloride-containing fluid). Additionally or alternatively, an analyte-selective substance could be disposed proximate one or both of the electrodes 135. In some examples, the body-mountable device 100 could include conductive polymers (e.g., polypyrrole) configured to provide electrical connections between the electrical components 140 and/or to provide one or more electrodes.

In some examples, electrochemical or other sensors could be provided as one or more of the electrical components 140 deposited on the metal traces 130, e.g., as the sensor 145. Sensor 145 could be a prefabricated electrochemical sensor, e.g., two or more electrodes formed on a flexible or rigid substrate that are subsequently disposed on the metal 130 traces of the body-mountable device 100. Such an electrochemical sensor 145 could be provided in this way due to a required geometry of the electrodes, a chemistry of the electrodes and/or the formation of the electrodes, or some other property of the electrodes or other components of the electrochemical sensor 145 that is not compatible with the composition and/or formation of the metal traces 130 and/or with some other process of formation of the body-mountable device 100. In some examples, the sensor 145 could be an optical sensor 145 configured to detect an optical property of the analyte-sensitive substance 160, of a fluid to which the analyte-sensitive substance 160 is exposed, an analyte in such a fluid, and/or of some other elements or substances that are related to a physiological property of interest.

Note that the windows 155, 157 formed in the encapsulating sealant layer 155 to provide access by the sensor 145 and electrodes 135 to a fluid are provided as a non-limiting example. In some examples, the encapsulating sealant layer 155 could be permeable to an analyte of interest such that the encapsulating sealant layer 155 could be formed to fully encapsulate the sensor 145 and/or electrodes 135. Further, the encapsulating sealant layer 155 could include an analyte-sensitive material (e.g., throughout the encapsulating sealant layer 155, within one or more specified regions of the encapsulating sealant layer 155 proximate the sensor 145 and/or electrodes 135) in addition to or instead of one or more distinct pieces or other elements formed of a separate analyte-sensitive material (e.g., 160). Further, an analyte-sensitive material could be formed over the encapsulating sealant layer 155, within a window or other formed feature of the encapsulating sealant layer 155, or otherwise formed or disposed according to an application.

The adhesive layer 120 and the encapsulating sealant layer 150 could include a variety of materials. In a preferred embodiment, one or both of the adhesive layer 120 and the encapsulating sealant layer 150 comprise a flexible material, e.g., a flexible polymeric material. The adhesive layer 120 and the encapsulating sealant layer 150 could be composed of the same material(s) or different materials. The adhesive layer 120 and/or the encapsulating sealant layer 150 could include silicones, polyurethanes, or other polymers, rubbers, or other flexible, elastomeric, or otherwise soft materials. The adhesive layer 120 and/or the encapsulating sealant layer 150 could include materials specified to resist or otherwise be resilient against damage incurred from an environment to which the body-mountable device 100 could be exposed. For example, the adhesive layer 120 and/or the encapsulating sealant layer 150 could be composed of materials that are resistant to damage, deterioration, cracking, depolymerization, or other processes caused by ultraviolet radiation, a high pH, a low pH, a biological environment, or some other environmental condition. Further, the materials of the encapsulating sealant layer 150 could be very soft and/or flexible materials (e.g., materials with a low Shore hardness) and/or could have a high adhesion (e.g., a high peel strength) to the electronics 140, 145, metal traces 130, and/or adhesive layer 120 such that the encapsulating sealant layer 150 could maintain adhesion to the electronics 140, 145, metal traces 130, and adhesive layer 120 when experiencing significant strain. In particular, the Shore hardness of the encapsulating sealant layer could be less than approximately 35, preferably between approximately 30 and approximately 35. The adhesion of the encapsulating sealant layer to the electronics 140, 145, metal traces 130, and/or adhesive layer 120 could provide a peel strength that is greater than approximately 30 pounds per square inch, preferably between approximately 30 pounds per square inch and approximately 55 pounds per square inch.

In a preferred embodiment, both the adhesive layer 120 and the encapsulating sealant layer 150 are composed of a silicone such that the metal traces 130, electronics 140, and/or other components of the body-mountable device 100 are encapsulated by an encapsulating, continuous layer of silicone. Such a silicone is preferably elastomeric, such that it is soft enough to experience strain without exerting sufficient internal stress to lose adhesion to the electronics 140, 145, metal traces 130, and/or adhesive layer 120 (e.g., such that it has a low Shore hardness). In some examples, the adhesive layer 120 and/or the encapsulating sealant layer 150 could have respective specified thicknesses, e.g., the adhesive layer 120 could have a thickness between approximately 2 microns and approximately 20 microns.

In some examples, the substrate 110 and adhesive layer 120 could be formed form the same material. In such examples, the substrate 110 and adhesive layer 120 could be formed as a single layer; that is, the substrate 110 and adhesive layer 120 could not be discrete from each other. For example, the substrate 110 and the adhesive layer 120 could be composed of a thermoplastic material (e.g., a liquid crystal polymer). In such examples, the thermoplastic material could be heated in order to adhere a metal foil to the thermoplastic material. Such a metal foil could then be modified (e.g., by etching) to form the metal traces 130.

As shown in FIGS. 1A and 1B, the body-mountable device 100 includes metal traces 130, electronic components 140, and other elements disposed on one side of the flexible substrate 110. However, embodiments as described herein could include devices wherein adhesive layers, metal traces, electronics, sensors, electrodes, encapsulating sealant layers, and/or other elements are disposed on both sides of a flexible substrate. Such devices could include vias or other formed elements to provide electrical connections through the flexible substrate (e.g., electrical connections between metal traces formed on opposite sides of the flexible substrate). An encapsulating sealant layer could be provided to wholly or partially encapsulate metal traces or other components disposed on one or both sides of a flexible substrate of such a body-mountable device. For example, electronics, sensors, electrodes, and other components could be disposed on a first side of a flexible substrate that is configured to be mounted to a skin surface (e.g., such that the sensors disposed on the first side of the flexible substrate can detect a physiological property of a user when the flexible substrate is mounted to the skin). A display, capacitive touch sensor electrodes, indicator lights, or other user interface elements could be disposed on a second side of the flexible substrate. An encapsulating sealant layer could be formed on both sides of such a device to fully encapsulate to user interface components on the second side of the flexible substrate and to partially encapsulate components on the first side of the flexible substrate (e.g., to encapsulate all of the metal traces and/or electronics except one or more electrodes or other elements of a sensor disposed on the first side of the flexible substrate).

A body-mountable device as described herein could include a user interface configured to provide a variety of functions and applications of the body-mountable device. In some examples, the user interface could provide means for changing or setting an operational state of the body-mountable device and/or for causing the performance of some function by the body-mountable device. For example, the user interface could provide means for a user to cause the body-mountable device to perform a measurement of a physiological property using a sensor, to set the body-mountable device into a sleep or other low-power state, to set a rate of operation of a sensor to detect a physiological property, or to control some other aspect of operation or function of the body-mountable device. In some examples, the user interface could provide means for inputting calibration or other data to the body-mountable device, e.g., for inputting calibration data related to the operation of a sensor to detect a physiological property. Additionally or alternatively, the user interface could provide means for inputting information about the state of a user of the body-mountable device, e.g., to indicate a physical or mental state of the user, to indicate an activity of the user, to indicate that the user has eaten a meal or taken a drug, or to indicate some other information. The user interface could provide means for providing an indication of information to a user, for example, information about the operation of the body-mountable device (e.g., battery charge state, an amount of free memory), detected physiological properties (e.g., a glucose level detected using a sensor), or some other information available to the body-mountable device.

An input component of a body-mountable device could be configured to detect a variety of inputs by detecting a variety of physical properties of the body-mountable device and/or of the environment of the body-mountable device. The input component could be configured to detect sound (e.g., voice commands), motion of the device (e.g., a gesture that includes motion of the skin surface to which the body-mountable device is mounted), contact between the body-mountable device and a finger or other portion of a user's body, or some other inputs. For example, the input component could be configured to detect a location, motion, pressure, gesture, or other information about objects (e.g., a finger or other body part) near the body-mountable device. The input component could include a capacitive touch sensor configured to detect a single touch, multiple touches, gestures, swipes, or other inputs. The input component could be and/or could include a flexible component (e.g., a capacitive touch sensor comprising one or more electrodes composed of one or more layers or sheets of a flexible conductive material and one or more sheets of a flexible nonconductive material). In some examples, the input component could include one or more elements in common with a sensor. For example, a sensor of the body-mountable device could be configured to detect a temperature of a skin surface to which the body-mountable device is mounted; additionally, the temperature sensor could be used to detect inputs (e.g., contact between the body-mountable device and a finger or other object) by detecting changes over time in the temperature detected using the temperature sensor.

An output component of a body-mountable device could be configured to provide indication of a variety of different types of information via a variety of means. The output component could provide an indication related to an operational status of the body-mountable device (e.g., to provide an indication related to the battery charge state or free memory space of the device, to provide an indication related to an operating mode or state of the device) and/or related to a physiological property detected using a sensor (e.g., to provide an indication related to a glucose level detected using a sensor). The output component could be used to provide an indication related to a course of action that a user could take (e.g., to administer a drug, to seek medical assistance). The output component could be used to provide an indication related to an alert generated by the body-mountable device (e.g., an alert that a measured physiological property is outside of some specified limits, and alert that a user is experiencing or is about to experience an adverse health state). The output component could include light-emitting elements (e.g., LEDs, OLEDs, displays), color-changing elements (e.g., e-ink elements or displays, LCDs), haptic elements (e.g., vibrators, buzzers, electrohaptic elements), acoustical elements (e.g., buzzers, speakers), or some other elements configured to provide an indication of some information, e.g., to a user. The output component could include flexible elements, e.g., the output component could include a flexible OLED display.

A body-mountable device as described herein could include a variety of sensors configured to detect a variety of physiological properties and/or properties of the environment of the body-mountable device. In some examples, the sensor could include an analyte sensor configured to detect an analyte (e.g., glucose) in a fluid on or within a skin surface or other body surface to which the sensing platform is mounted and/or otherwise placed into contact with (e.g., interstitial fluid within or beneath the skin). In such examples, the sensor could include two or more electrodes configured to detect the analyte electrochemically (e.g., potentiometrically or amperometrically using, e.g., electrodes 135), optically (e.g., by illuminating and/or detecting light emitted from an analyte-sensitive substance, e.g., 160, that has an optical property related to the analyte), or by some other means. One or more sensors could detect a temperature on or within skin or some other environment to which the device is exposed. One or more sensors could be configured to detect an electrical or magnetic field, an electrical potential between two points on or within skin or some other environment to which the device is exposed (e.g., to detect an electromyogram, to detect an electrocardiogram, to detect a galvanic skin potential), an electrical conductivity between two or more points (e.g., to detect a galvanic skin response, to detect a skin conductance), or some other electrical and/or magnetic property or variable on or within skin and/or in the environment of the device. One or more sensors could be configured to detect and/or emit light, e.g., to illuminate and/or detect light emitted from on or within skin or other tissue (e.g., to photoplethysmographically detect a flow of blood within the skin and/or to detect a timing and/or rate of heartbeats), to detect ambient light received by the device (e.g., to detect the presence, motion, or other properties of a finger or other body part proximate the device, e.g., to receive an input from a user). Additional or alternative sensors detecting additional or alternative properties or variables are anticipated.

The sensor could be disposed on a sensor probe (not shown) that is configured to penetrate skin or other tissue (e.g., to a specified depth within skin) such that the sensor can measure an analyte in a fluid within the skin or other tissue. Such a sensor probe could be configured to penetrate to a specified depth within the tissue (e.g., to a depth within the dermis, to a subcutaneous depth) such that at least one sensor disposed on the sensor probe can measure an analyte in fluid (e.g., interstitial fluid) at the specified depth. The sensor probe could be flexible or rigid; in some examples, the sensor probe could comprise an elongate extension of the flexible substrate material 110. The sensor probe could be configured to pierce the skin or other tissue (e.g., could be sufficiently rigid and/or sharpened such that the sensor probe can be driven into the skin). Additionally or alternatively, the sensor probe could be configured to pierce and/or penetrate the skin or other tissue in combination with an insertion device. For example, the sensor probe could be configured to be mounted within the channel of a half-needle or to some other means for piercing the tissue; the half needle or other piercing means could be used to pierce the tissue and to subsequently retract, leaving the sensor probe in place penetrating the tissue. One or more sensors could be disposed at the end of such a sensor probe and/or at one or more additional locations along the length of such a sensor probe.

A body-mountable device as described herein (e.g., 100) can include a power source, electronics, and an antenna all disposed on a flexible substrate configured to be mounted to skin of a living body or to be mounted or otherwise disposed proximate to some other surface or tissue of a body or other environment of interest. The electronics can operate one or more sensors (e.g., a sensor disposed at the distal end of a sensor probe) to perform measurements of an analyte (e.g., to measure the concentration of the analyte in interstitial fluid within or beneath the skin) or some other components to perform some other functions (e.g., receiving a user input, detecting a property of the environment of the device) according to an application. The electronics could additionally operate the antenna to wirelessly communicate the measurements from the sensor or other information to an external reader or to some other remote system via the antenna. One or more of the power source, antenna, electronics, or other components of the device could be flexible; for example, the power source could include a thin, flexible lithium ion battery. In some examples, one or more of the power source, antenna, electronics, or other components of the device could be sufficiently flexible to allow for flexibility of the overall device and/or of elements of the device that are able to be mounted to and/or within skin or other tissue (e.g., to provide greater comfort and/or to minimize effect on user activities when mounted to and/or within skin or other tissues of a user).

Batteries of a sensing platform as described herein could be single-use or could be rechargeable. Rechargeable batteries could be recharged by power provided by radio frequency energy harvested from an antenna disposed on the flexible substrate. The antenna can be arranged as a loop of conductive material with leads connected to the electronics. In some embodiments, such a loop antenna can also wirelessly communicate the information (e.g., measurements of the analyte made using a sensor of the sensing platform) to an external reader (e.g., to a cellphone) by modifying the impedance of the loop antenna so as to modify backscatter radiation from the antenna. Additionally or alternatively, the sensing platform could include a chip, dipole, or other type of antenna for transmitting and/or reflecting RF energy to indicate information to an external reader. Further, such antennas could be used to transfer additional information, e.g., to indicate a temperature, light level, or other information detected by the sensing platform, to receive commands or programming from an external device, or to provide some other functionality.

Note that, while embodiments described herein are generally described as configured to mount to a body surface and/or to be otherwise used while in proximity to part of a human or animal body to detect properties of such bodies and/or to perform some other functions, the embodiments described herein could be used in other contexts to perform other functions. For example, embodiments described herein could provide flexible sensing platforms or other flexible electronic devices configured to operate in corrosive fluids, conductive fluids, humid environments, or environments that could otherwise affect the operation of electronics, e.g., by providing shorting conductive paths between elements (e.g., metal traces) of electronics, by corroding or otherwise degrading elements of electronics (e.g., by corroding metal traces, integrated circuit pads, or other metallic elements of electronics). Such embodiments provide such functionality by wholly or partially encapsulating metal traces, integrated circuit packages, antennas, batteries, or other components within an encapsulating layer of a sealant, e.g., within an encapsulating layer of silicone (e.g., elastomeric silicone) adhesive. Such embodiments could further include such components being disposed on a layer of adhesive (e.g., a layer of silicone adhesive) on which the encapsulating sealant layer is also formed and to which such a sealant layer adheres. Such flexible electronic devices could be configured to detect one or more properties (e.g., a concentration of an analyte in a fluid) using one or more sensors, to identify the flexible electronic devices and/or object to which such devices are mounted (e.g., via an RFID or other wireless communications protocol), or to provide some other functions in a natural environment (e.g., a marsh, a lake, a stream), an artificial environment (e.g., a water treatment process, the hold of a container ship), an environment of a pharmaceutical synthesis process (e.g., within fluid of a reactor vessel), a food processing environment (e.g., within a mixing vessel), or some other environment.

It should be understood that the above embodiments, and other embodiments described herein, are provided for explanatory purposes, and are not intended to be limiting.

II. Example Fabrication of a Body-Mountable Device

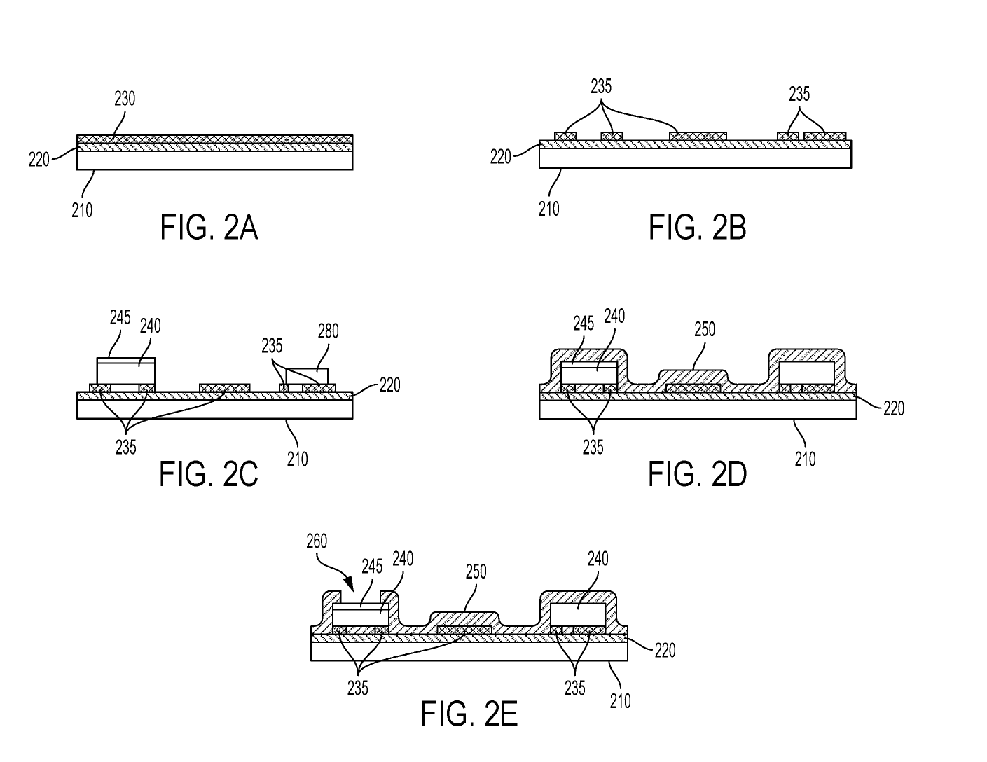

A body-mountable device as described herein could be formed by a variety of processes. FIGS. 2A-E show, in cross-section, elements of a body-mountable device as the device is fabricated. The illustrated process of fabrication of a body-mountable device, and any steps or methods described in connection with the execution of such a process, are intended as illustrative examples of the fabrication of a body-mountable device and are not intended to be limiting.

FIG. 2A shows, in cross-section, a flexible substrate 210. On the flexible substrate is disposed an adhesive layer 220 on which is disposed a layer of metal 230. These elements could be formed by deposition of the adhesive layer 220 on the flexible substrate 210, the deposition of the metal layer 230 on the adhesive layer 220, the formation of the flexible substrate 210 on the adhesive layer 220, and/or the deposition of the adhesive layer 220 on the layer of metal 230 by one or more processes. Such processes could include spray coating, spin coating, chemical vapor deposition, physical vapor deposition, curing (e.g., thermal curing, ultraviolet curing), or some other deposition process. In some examples, one or more of the layers (e.g., 210, 220, 230) could be provided as sheets and the one or more layers could be pressed together or otherwise formed (e.g., using an adhesive, e.g., the adhesive layer 220 and/or an adhesive that can be formed into the adhesive layer 220) into the structure shown in FIG. 2A. For example, the metal layer 230 and flexible substrate 210 could be provided as sheets of material (e.g., a metal foil and a sheet of polyimide, polyethylene terephthalate, or some other polymer, rubber, or other flexible material) to which an adhesive could be applied (e.g., by spraying, by dipping, by spin-coating) such that the metal layer 230 and flexible substrate 210 could be adhered together by an adhesive layer 220 as shown in FIG. 2A. In some examples, the adhesive layer 220 could be formed from a layer of pressure-sensitive adhesive (e.g., the adhesive layer 220 could be formed from a sheet of a silicone that includes tackifying elements or that has otherwise been configured to be pressure-sensitive) and structure shown in FIG. 2A could be formed by pressing a metal foil and the flexible substrate 210 against opposite sides of the adhesive layer 220.

In some examples, the adhesive layer 220 could be composed of a thermoplastic material (e.g., a liquid crystal polymer). In such examples, the thermoplastic material could be heated in order to adhere the metal foil (forming the metal layer 230) to the thermoplastic material. This could take the form of a sheet of thermoplastic material being heated to adhere to a metal foil. Alternatively, the thermoplastic material could be applied, as a melt, to the metal foil (e.g., via spin-coating) to form the adhesive layer 220 on the metal layer 230. In some examples, the substrate 210 and adhesive layer 220 could be formed form the same material (e.g., the same thermoplastic material). In such examples, the substrate 210 and adhesive layer 220 could be formed as a single layer; that is, the substrate 210 and adhesive layer 220 could not be discrete from each other.

FIG. 2B shows, in cross-section, the flexible substrate 210 and adhesive layer 220 of FIG. 2A after the metal layer 230 has been formed into metal traces 230. The formed metal traces 235 are configured (e.g., have a shape, size, thickness, composition, conductivity) to provide electrical connections between one or more electronic components. The metal layer 230 could be formed into the metal traces 230 by a variety of methods. In some examples, a photoresist could be applied and patterned using light to expose regions of the metal layer 230 that could be subsequently removed (e.g., by a chemical etch process) to leave behind unexposed regions of the metal layer 230 that form the metal traces 235. In some examples, a laser, ion beams, engraver, or other means could be used to remove regions of the metal layer 230 to form the metal traces 235. Further, forming the metal traces 235 from the layer of metal 230 could include forming antennas, electrochemical and/or electrophysiological electrodes, electrical contact pads, capacitive touch sensor electrodes, or other shapes or structures.

FIG. 2C shows, in cross-section, the flexible substrate 210, adhesive layer 220, and metal traces 235 of FIG. 2B after electronics (e.g., a controller 280, a sensor 240) have been disposed on the metal traces 235. The electronics 204, 280 could be disposed on the metal traces by pick-and-place machines, by self-assembly, or by some other method. Disposing the electronics 240, 280 on the metal traces 235 could include electrically connecting attaching the electronics 240, 280 to the metal traces 235, e.g., by soldering, by applying pressure between the metal traces 235 and the electronics 240, 280, by wire-bonding, by use of a conductive material (e.g., a conductive liquid crystal, a conductive epoxy), by reflowing the metal traces 235, or by some other means.

An analyte-sensitive substance 245 is disposed on a sensor 240 of the electronics. The analyte-sensitive substance 245 is a substance (e.g., a protein, a fluorophore, an aptamer, an antibody, an ionophore) that selectively interacts with (e.g., binds to, complexes with, reacts with, catalyzes a reaction of) an analyte or other constituent of a fluid (e.g., a protein, a cell, an ion) or other environment of interest such that the sensor 240 can detect the analyte of interest (e.g., optically, electrochemically). Additionally or alternatively, the metal traces 235 could form one or more electrodes (e.g., by being electroplated with gold, silver, platinum, or some other substance, by being electrochemically activated by oxidation, or by some other process) and such an analyte-sensitive substance could be disposed proximate such formed electrodes. Such an analyte sensitive-substance could be disposed on the sensor 240 or on some other component prior to disposing the sensor 240 or other component on the metal traces. Additionally or alternatively, the analyte sensitive-substance could be disposed on the sensor 240 or on some other component after disposing electronics on the metal traces, after forming the metal traces 235 from the metal layer 230, before forming the metal traces 235 from the metal layer 230, or at some other point in the process of fabricating a body-mountable device as shown in FIGS. 2A-E.

FIG. 2D shows, in cross-section, the flexible substrate 210, adhesive layer 220, metal traces 235, analyte-sensitive substance 245, and electronics 240, 280, of FIG. 2C after an encapsulating sealant layer 250 has been formed. The encapsulating sealant layer 250 is formed to adhere to the adhesive layer 220 and to at least partially encapsulate the metal traces 235 and the electronics 240, 280, e.g., by coating and adhering to surfaces of the metal traces 235 and the electronics 240, 280. This could include the encapsulating sealant layer 250 coating and adhering to surfaces of the metal traces 235 and the electronics 240, 280 such that substantially no voids or volumes exist proximate to surfaces of the metal traces 235 and electronics 240, 280 in which water vapor or other material (e.g., material that has permeated through the encapsulating sealant layer 250 from a fluid to which the encapsulating sealant layer 250 is exposed) could be deposited, condense, or otherwise accumulate.

In some examples, the encapsulating sealant layer 250 could be formed as a conformal sealant layer, having a substantially uniform thickness across those surfaces on which the sealant layer is disposed (e.g., across surfaces of the adhesive layer 220, metal traces 235, analyte-sensitive substance 245, and electronics 240, 280 that are encapsulated by the encapsulating sealant layer 250). Such a conformal sealant layer could be formed to have a specified thickness, e.g., the conformal sealant layer could have a thickness between approximately 5 microns and approximately 200 microns. In some examples, the encapsulating sealant layer 250 could be formed as a planarizing sealant layer, having a substantially uniform height relative to the adhesive layer 220 across those surfaces on which the sealant layer 250 is disposed. The encapsulating sealant layer 250 could be configured in some other way.

Forming the encapsulating sealant layer 250 could include depositing a precursor material (e.g., a solution, vapor, or other material including monomers or other chemicals from which a silicone, rubber, or other material of the encapsulating sealant layer 250 could be formed) onto the adhesive layer 220, metal traces 230, electronics 240, 280, and analyte-sensitive material 245. Such a precursor material could include a liquid silicone adhesive rubber, e.g., Dow Corning 3140. In some examples, the precursor could be a photo-patternable liquid silicone adhesive rubber , e.g., Dow Corning WL-5150, to provide means for creating windows through the encapsulating sealant layer 250 through lithography or other methods, e.g., such that the analyte-sensitive substance 245 or some other sensor could be exposed. Depositing the precursor material could include spraying the precursor material, dipping the flexible substrate 210 and elements attached thereto into the precursor material, applying the precursor material via chemical vapor deposition or physical vapor deposition, applying the precursor material via a spin-coating process (e.g., such that the formed sealant layer forms a planarizing layer having a specified uniform height relative to the adhesive layer 220), or applying the precursor material via some other process. Forming the encapsulating sealant layer 250 could additionally include curing such a deposited precursor material, e.g., by exposing the precursor material to a controlled temperature, by exposing the precursor material to a controlled humidity, by exposing the precursor material to ultraviolet radiation, by exposing the precursor material to a curing agent (e.g., a polymerization initiator), or by some other means. Forming the encapsulating sealant layer 250 could include further steps. In some examples, forming the encapsulating sealant layer 250 could include exposing the flexible substrate 210 and component disposed thereon to an agent configured to increase the adhesion of the encapsulating sealant layer 250 to such components (e.g., by exposure to an adhesive material, by exposure to a cleaning solution or cleaning plasma).

FIG. 2E shows, in cross-section, the flexible substrate 210, adhesive layer 220, metal traces 235, analyte-sensitive substance 245, electronics 240, 480, and encapsulating sealant layer 250 of FIG. 2D after at least one window 260 has been formed in the encapsulating sealant layer 250. As a result, the encapsulating sealant layer 250 does not encapsulate the analyte-sensitive substance 245 disposed on the sensor 240. The window 260 could be formed in a variety of ways. In some examples, the window 260 could be formed by ablating a portion of the encapsulating sealant layer 250 using, e.g., a laser, an ion beam, an abrasive and/or cutting tool, or by some other means. For example, the encapsulating sealant layer 250 could be composed of a silicone and could be ablated using a laser having a wavelength specified to be absorbed by bonds of the silicone, e.g., the laser could be a short-pulse laser configured to emit light at approximately 355 nanometers.

In some examples, the window 260 could be formed by chemically etching a portion of the encapsulating sealant layer 250. Etching could include forming a resist to protect regions of the encapsulating sealant layer 250 that are not the region(s) to be etched, e.g., by exposure to a chemical bath, exposure to a plasma, or exposure to some other etching means. Etching could include dissolving or otherwise etching portion(s) of the encapsulating sealant layer 250 that have been specified, e.g., during the formation of the encapsulating sealant layer 250. For example, the encapsulating sealant layer could be formed from a photo-patternable precursor material (e.g., Dow Corning WL-5150) and regions of the encapsulating sealant layer 250 that are not the region(s) to be etched could be exposed to ultraviolet light or other radiation to e.g., begin crosslinking and/or polymerization of the photo-patternable material; subsequently, regions of the encapsulating sealant layer 250 that were not cured in this way could be etched, e.g., by washing off the un-exposed portions of the encapsulating sealant layer 250.

Note that, while the analyte-sensitive substance 245 is shown as being disposed on a sensor 240 prior to formation of the encapsulating sealant layer 250, such a substance could be disposed as part of a step that is prior to or subsequent to such a step. For example, the analyte-sensitive substance could be disposed on electrodes formed from and/or deposited or otherwise formed on the metal traces 235 prior to the electronics 240, 280 being disposed on the metal traces 235. In some examples, an analyte-sensitive substance could be disposed within a window (e.g., 260) formed in the encapsulating sealant layer 250 subsequent to the formation of such a window (e.g., by being deposited as a liquid in the formed window and subsequently, e.g., cured, polymerized, crosslinked, dried, or otherwise formed into the analyte-sensitive material). Further, an analyte-sensitive substance could be disposed on a surface of the encapsulating sealant layer 250, e.g., opposite the encapsulating sealant layer 250 from an optical sensor configured to detect on optical property of the analyte-sensitive substance (e.g., by emitted illumination and detecting responsively emitted fluorescent light) that is related to a concentration or other property of the analyte to which the analyte-sensitive substance is exposed.

A process to fabricate a body-mountable device as described in relation to FIGS. 2A-E could include additional steps. For example, such a process could include controlling a shape of the body-mountable device by cutting the flexible substrate 210 and/or other elements of the device into a specified shape using a laser, a stamp, or some other means. In some examples, one or more of the body-mountable device could be formed on a sheet of the flexible substrate 210 and specifying the shape of the body-mountable device could include cutting the body-mountable device from such a sheet of flexible material (e.g., by using a laser, stamp, or other means to cut the flexible substrate into a specified shape). Such a specified shape could be specified according to an application. In some examples, the specified shape could include an elongate portion on which a sensor (e.g., an electrochemical sensor including two or more electrodes) is disposed and that is configured to penetrate skin or other tissue. In some examples, such a specified shape could be a ring, circle, arc, or other shape specified to be at least partially embedded in an ophthalmic lens that is, e.g., configured to be mounted to an eye such that a sensor of the formed body-mountable device could be exposed to tears of the eye and/or such that the formed body-mountable device could perform some functions related to the eye. In some examples, the adhesive layer 220 and/or the substrate 210 could include a thermoplastic material (e.g., a liquid crystal polymer) and the process could further include one or more steps to anneal such a thermoplastic material by applying heat to the thermoplastic material. Such annealing could be performed to reduce curling of the substrate 210 and/or adhesive layer 220, to reduce a stress in the substrate 210 and/or adhesive layer 220, or to provide some other benefit.

Note that, while the body-mountable device formed in FIGS. 2A-E includes an adhesive layer, metal traces, electronics, and a encapsulating sealant layer disposed on a single side of a flexible substrate, a body mountable device as described herein could include one or more of an adhesive layer, metal traces, electronics, and a encapsulating sealant layer disposed on both sides of a flexible substrate. In such examples, vias or other elements could be include to provide an electrical connection between metal traces, electronic components, sensors, or other elements on opposite sides of the flexible substrate. Further, a body-mountable device could include multiple layers of metal traces or other elements disposed on a single side of a flexible substrate. In some examples, the substrate 210 and adhesive layer 220 could be formed from the same material and/or could form a single continuous element.

In some examples, a plurality of body-mountable device could be fabricated from a roll of material, e.g., a roll of a flexible substrate, a roll of a flexible substrate to which a metal layer is adhered by an adhesive layer, or a roll of some other material. In such examples, one or more processes for fabricating the plurality of body-mountable devices could be performed by unrolling such a roll of material, performing the one or more processes, and rolling the plurality of partially formed body-mountable devices on the material onto a second roll. Further processing to fabricate the plurality of body-mountable devices could include unrolling the second roll of partially formed body-mountable devices on the material and performing one or more further processes on the partially formed body-mountable devices.

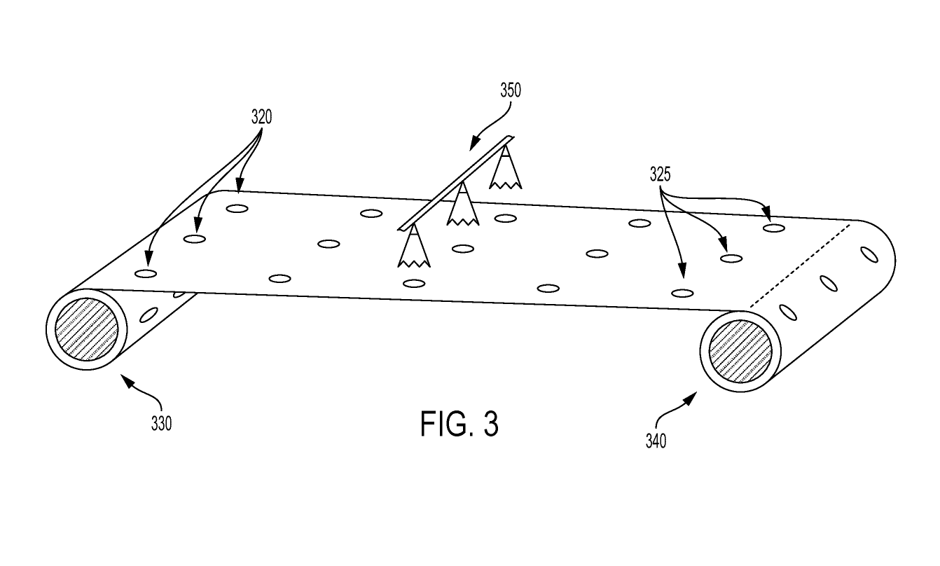

FIG. 3 illustrates such a roll-to-roll fabrication process. A first roll 330 includes a first plurality of partially-formed body-mountable devices 320. The material (e.g., flexible substrate material) of the first roll 330 on which the first plurality of devices 320 is disposed is taken off of the first roll 330 and passed through a processing station 350. The processing station 350 performs one or more fabrication steps to the first plurality of devices 320, forming a second plurality of partially-formed body-mountable devices 325. The second plurality of partially-formed body-mountable devices 325 and the material (e.g., flexible substrate material) on which the first plurality of devices 325 is disposed is rolled onto a second roll 340.

As shown in FIG. 3, the processing station 350 includes sprayers configured to coat the first plurality of partially-formed body-mountable devices 320 with a material, e.g., with a precursor material configured to form into a encapsulating sealant layer, a liquid configured to form an analyte-sensitive substance. However, the processing station 350 could include additional or alternative systems or devices configured to perform additional or alternative processing steps on the first plurality of partially-formed body-mountable devices 320. In some examples, the processing station 350 could include photolithographic systems, etching systems, ablative lasers, or other means for patterning a metal layer to form metal traces (e.g., metal traces configured to provide electrical connections between electronic components, metal traces configured to act as electrodes, e.g., of an electrochemical sensor, of a capacitive touch sensor). In some examples, the processing station 350 could include one or more pick-and-place machines, infrared reflow ovens, or other means for disposing electronics or other components on metal traces formed on the flexible substrate. In some examples, the processing station 350 could include chemical etchants, ablative lasers, or other means for forming one or more windows in a formed encapsulating sealant layer. In some examples, the processing station 350 could include cutting lasers, stamps, or other means for cutting the plurality of body-mountable devices disposed on the flexible material into specified shapes. The processing station 350 could include additional or alternative means for performing additional or alternative processing steps on partially-formed body-mountable devices.

In some examples, one or more processing steps could be performed on partially-formed body-mountable devices related to the roll-to-roll processing of the body-mountable devices. For example, the body-mountable devices could be formed from a sheet of material comprising a layer of metal adhered to a flexible substrate by an adhesive layer. In such examples, the metal layer could be etched or otherwise patterned to form metal interconnects. In such examples, a tackiness of the adhesive layer exposed by removal of portions of the metal layer to form the metal traces could be reduced, e.g., by exposing the adhesive layer to an oxygen plasma or some other material. Additionally or alternatively, the adhesive layer could be composed of a material that has a low tackiness.

III. Example methods

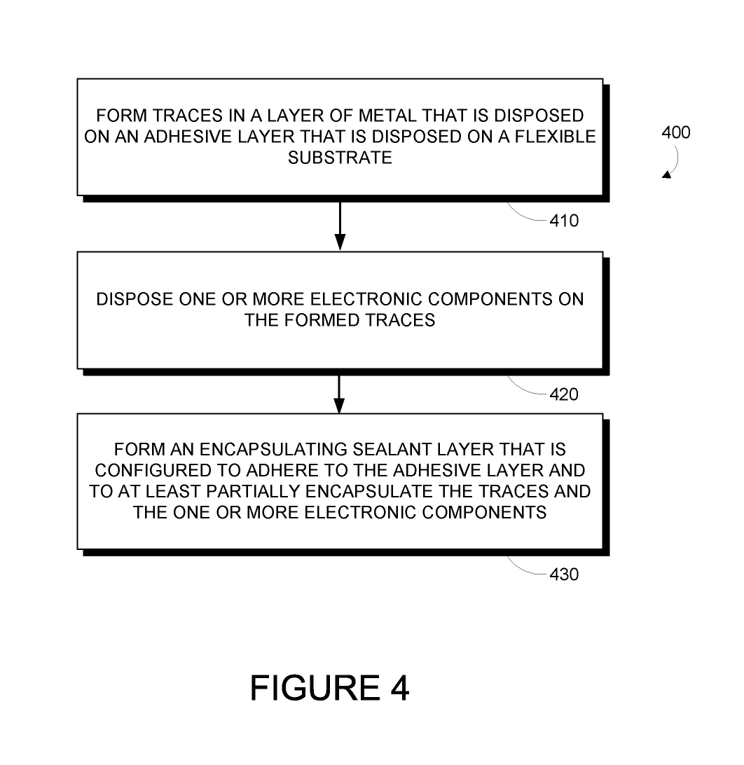

FIG. 4 is a flowchart of a method 400 for fabricating a device. The method 400 includes forming traces in a layer of metal that is disposed on an adhesive layer that is disposed on a flexible substrate (410). This could include using photolithographic, stamping, or other techniques to form a patterned resist (e.g., a dry laminate resist like a Dupont Riston dry resist) on the metal layer and subsequently to etch traces into the metal layer using the formed resist, e.g., using a chemical etchant, a plasma or ion etch, or some other etching means. Forming traces in the metal layer (410) could include using an ion beam, laser, or other directed energy to ablate regions of the metal layer to from traces. Forming traces in the metal layer (410) could include forming antennas, electrodes (e.g., electrochemical electrodes, electrophysiological electrodes, capacitive touch sensing electrodes), elements of a display (e.g., electrodes of an LCD, e-paper, or other display), or other structures from the metal layer. In examples, wherein forming traces in the metal layer (410) includes forming electrochemical electrodes, the method 400 could further include forming an electrochemical interface layer on the electrodes (e.g., a layer composed of a specified material or materials, e.g., gold, platinum, silver, silver-chloride, titanium, steel, polypyrrole or other conductive polymers or other metals or materials) by electroplating, chemical vapor deposition, physical vapor deposition, spraying, dipping, spin-coating, curing, or some other process or combination of processes.

The method 400 additionally includes disposing one or more electronic components on the formed traces (420). This could include manually disposing electronic components, using a pick-and-place machine to position the electronics, using self-assembly to position the electronics, or some other means or methods to dispose one or more electronic components at respective different locations on the formed traces and further electrically connecting such electronic devices to the traces. Disposing one or more electronic components on the formed traces (420) could include disposing a flux, solder, anisotropic conductive material, conductive epoxy, or other materials on the metal traces such that the electronics could be electrically connected to the metal traces, e.g., by reflowing a solder, by curing a conductive epoxy, or by some other means or methods.

The method 400 additionally includes forming an encapsulating sealant layer that is configured to adhere to the adhesive layer and to at least partially encapsulate the traces and the one or more electronic components (430). This could include depositing a precursor material (e.g., a liquid silicone adhesive rubber, e.g., Dow Corning 3140) on the adhesive layer, metal traces and/or one or more electronic components and subsequently curing the deposited precursor material. Depositing a precursor material could include spraying, dipping, spin-coating, or depositing the precursor material by some other means or methods. Curing the deposited precursor material could include drying the material, exposing the material to ultraviolet radiation or some other energy, exposing the material to an environment having a specified temperature, humidity, or other specified property, exposing the material to a curing agent (e.g., a radical polymerization initiator), or forming the deposited precursor material into a conformal encapsulating sealant layer, a planarizing, encapsulating sealant layer, or some other form of encapsulating sealant layer by some other means or methods.

The method 400 could include additional steps or elements in addition to those illustrated in FIG. 4. For example, the method 400 could include forming one or more sensors on the flexible substrate. Forming sensors could include disposing electronics (e.g., light emitters, light detectors, transducers, electrochemical electrodes formed on a discrete substrate), forming electrodes from the metal layer and/or from formed traces on the adhesive layer, disposing analyte-sensitive materials, or disposing or forming other elements on the flexible substrate and/or on some other elements of a device formed by the method 400. Such formed sensors can include analyte-sensitive material, i.e., material that selectively interacts with (e.g., binds to, complexes with, reacts with, catalyzes a reaction of) an analyte (e.g., an ion, a cell, a protein, an antibody, a chemical) and the method 400 could include depositing or forming such an analyte sensitive material, e.g., on an electrode, proximate an optical fiber, proximate a light sensor, or according to some other application. The method 400 could include forming windows or other accesses into/through a formed encapsulating sealant layer, e.g., to expose electrodes, analyte-sensitive materials, or other components of sensors to an environment of a device formed by the method 400. This could include ablating regions of a formed encapsulating sealant layer (e.g., using a laser, using an ion beams, using an ablative tool), selectively curing regions of a deposited precursor material of the encapsulating sealant layer (e.g., by selectively exposing regions of a photo-patternable precursor material to light), or using some other means or methods. In some examples, the adhesive layer and/or the substrate could include a thermoplastic material (e.g., a liquid crystal polymer) and the method 400 could further include one or more steps to anneal such a thermoplastic material by applying heat to the thermoplastic material. Such annealing could be performed to reduce curling of the substrate and/or adhesive layer, to reduce a stress in the substrate and/or adhesive layer, or to provide some other benefit.

The method 400 could include cutting the flexible substrate and/or other elements of a device formed via the method 400 into a specified shape, e.g., using a laser, stamp, or other means or methods. Cutting the device or elements thereof into a specified shape could include forming the device into ring, disc, or other shape. In some examples, the specified shape could be specified to allow the flexible substrate to be mounted to a skin surface or to the surface of some other tissue of a body. In some examples, the specified shape could be specified to allow the flexible substrate to be incorporated into some other components/devices/systems, e.g., to be partially embedded into an ophthalmic lens that is configured to be mounted to a surface of an eye.

One or more of the steps of the method 400 could be performed as part of a roll-to-roll fabrication process. For example, a plurality of devices could be formed on a single sheet of a flexible substrate (or other material that is capable of being rolled and unrolled) and processed as a group. For example, a group of partially-formed devices disposed on a roll of a flexible substrate material could be unrolled, an encapsulating sealant layer precursor could be sprayed or otherwise deposited on the group of partially-formed devices and could be subsequently cured (e.g., by exposure to heat), and the group of partially-formed devices with encapsulating sealant layers thusly formed thereon could be rolled onto a second roll and/or subjected to further processing steps. In such examples, the method 400 could include additional steps related to the roll-to-roll process, e.g., the method 400 could include reducing a tackiness of regions of the adhesive layer that are exposed by forming traces from the metal layer. Reducing the tackiness of exposed regions of the adhesive layer could include exposing the adhesive layer to an oxygen plasma or some other material. Additionally or alternatively, the adhesive layer could be composed of a material that has a low tackiness. Additional and/or alternative steps of the method 1100 are anticipated.

What is claimed is:

1. A method comprising:
forming traces in a layer of metal, wherein the layer of metal is disposed on an adhesive layer, wherein the adhesive layer is disposed on a flexible substrate, and wherein forming the traces comprises patterning the layer of metal to provide electrical connections between one or more electronic components;
disposing one or more electronic components on the traces, wherein disposing the one or more electronic components on the traces comprises electrically connecting the one or more electronic components to the traces; and
forming an encapsulating sealant layer, wherein forming an encapsulating sealant layer comprises forming a sealant layer that is configured to adhere to the adhesive layer and to at least partially encapsulate the traces and the one or more electronic components.

2. The method of claim 1, wherein the encapsulating sealant layer comprises elastomeric silicone.

3. The method of claim 2, wherein the adhesive layer comprises elastomeric silicone.

4. The method of claim 1, wherein forming an encapsulating sealant layer that is configured to at least partially encapsulate the traces and the one or more electronic components comprises forming an encapsulating sealant layer that includes at least one window, and further comprising:
forming at least one window in the formed encapsulating sealant layer by laser-ablating material of the formed encapsulating sealant layer.

5. The method of claim 1, wherein forming an encapsulating sealant layer that is configured to at least partially encapsulate the traces and the one or more electronic components comprises forming an encapsulating sealant layer that includes at least one window, and further comprising:
forming at least one window in the formed encapsulating sealant layer by chemically etching material of the formed encapsulating sealant layer.

6. The method of claim 1, wherein forming an encapsulating sealant layer comprises:
spraying a precursor material onto the adhesive layer, traces, and one or more electronic components; and
curing the sprayed precursor material.

7. The method of claim 1, further comprising:
cutting the flexible substrate into a specified shape using a laser.

8. The method of claim 1, further comprising:
exposing the adhesive layer, subsequent to forming the traces, to an oxygen plasma to reduce a tackiness of portions of the adhesive layer that are exposed by forming the traces.

9. The method of claim 1, wherein the substrate and the adhesive layer are formed from the same material, wherein the substrate and the adhesive layer comprise a thermoplastic material, the method further comprising:
adhering the adhesive layer to a metal foil, wherein adhering the adhesive layer to the metal foil comprises applying heat to the thermoplastic material of the substrate and adhesive layer, and wherein forming traces in a layer of metal comprises forming traces from the metal foil.

10. The method of claim 9, further comprising, subsequent to adhering the metal foil to the adhesive layer, annealing the thermoplastic material of the substrate and adhesive layer, wherein annealing the thermoplastic material comprises applying heat to the thermoplastic material.

11. The method of claim 1, wherein forming an encapsulating sealant layer comprises forming a conformal sealant layer that is configured to adhere to the adhesive layer and to at least partially encapsulate the traces and the one or more electronic components.

12. The method of claim 1, wherein forming an encapsulating sealant layer comprises forming a sealant layer that adheres to the metal traces and to the one or more electronic components with a peel strength between approximately 30 pounds per inch and approximately 55 pounds per inch.

13. The method of claim 1, wherein forming an encapsulating sealant layer comprises forming a sealant layer having a Shore hardness less than approximately 35.

14. A body-mountable device comprising:
a flexible substrate;
an adhesive layer disposed on the flexible substrate;
one or more electronic components disposed on the adhesive layer;
metal traces disposed on the adhesive layer, wherein the metal traces provide electrical connections to the one or more electronic components; and
an encapsulating sealant layer, wherein the encapsulating sealant layer is configured to adhere to the adhesive layer and to at least partially encapsulate the metal traces and the one or more electronic components.

15. The body-mountable device of claim 14, wherein the encapsulating sealant layer comprises elastomeric silicone.

16. The body-mountable device of claim 15, wherein the substrate and the adhesive layer are formed from the same material and wherein the substrate and the adhesive layer comprise a thermoplastic material.

17. The body-mountable device of claim 14, further comprising:
an electrochemical sensor, wherein the electrochemical sensor is disposed on the flexible substrate, wherein the electrochemical sensor comprises at least two electrodes, and wherein the encapsulating sealant layer does not encapsulate the at least two electrodes of the electrochemical sensor.

18. The body-mountable device of claim 14, wherein the encapsulating sealant layer comprises a conformal sealant layer.

19. The body-mountable device of claim 14, further comprising:
an ophthalmic lens, wherein the flexible substrate is at least partially embedded in the ophthalmic lens.

20. The body-mountable device of claim 14, wherein the encapsulating sealant layer adheres to the metal traces and to the one or more electronic components with a peel strength between approximately 30 pounds per inch and approximately 55 pounds per inch.

21. The body-mountable device of claim 14, wherein the encapsulating sealant layer has a Shore hardness less than approximately 35.
